(19) United States
(12) Patent Application Publication
(10) Pub. No.: US 2024/0358983 A1
(43) Pub. Date: Oct. 31, 2024

(54) Systems And Methods For Shockwave Mediated Delivery Of Microvesicles And Stem Cells

(71) Applicant: Synaptec Network, Inc., Santa Monica, CA (US)

(72) Inventor: Sheldon Jordan, Pacific Palisades, CA (US)

(21) Appl. No.: 18/646,463

(22) Filed: Apr. 25, 2024

Related U.S. Application Data
(60) Provisional application No. 63/462,333, filed on Apr. 27, 2023.

(51) Int. Cl. A61M 37/00

(52) U.S. Cl. CPC A61M 37/00; A61M 2202/0007; A61M 2202/0437; A61M 2202/06; A61M 2202/08; A61M 2210/0693

(57) ABSTRACT

Methods, systems, and devices are disclosed for facilitating delivery of microvesicles or stem cells across the blood brain barrier (BBB). A pressure device is used to target a region of a patient's brain. The pressure device delivers a shockwave to the targeted region, facilitating delivery of a microvesicle or stem cell at the targeted region. The shockwave does not damage brain tissue and does not disrupt or tear the BBB. A microvesicle or stem cell, or both, is administered to the patient and crosses the BBB at the targeted region, without damaging, tearing, or disrupting the BBB.

This application claims priority to U.S. provisional application No. 63/462,333, filed Apr. 27, 2023, the disclosure of which is incorporated herein by reference.

### FIELD OF THE INVENTION

The field of the invention is methods, systems, kits, and devices related to delivering macrovesicles and stem cells to the brain.

### BACKGROUND

The background description includes information that may be useful in understanding the present invention. It is not an admission that any of the information provided herein is prior art or relevant to the presently claimed invention, or that any publication specifically or implicitly referenced is prior art.

In some cases, the most desirable way to treat an ailment is to treat the source. However, for treatment of ailments in or relating to regions of the brain, the blood-brain barrier (BBB) often hinders treatment by preventing diffusion of therapeutic agents into the brain. While it is known to mechanically bypass the BBB, for example through use of a needle to inject therapeutic agents directly into the brain or otherwise tear through the tight intercellular junctions of the BBB, such methods are undesirable due to damage caused by such invasive methods.

More sophisticated methods of delivering therapeutic agents to regions of the brain are known. For example, “Targeted Delivery of Neural Stem Cells to the Brain Using MRI-Guided Focused Ultrasound to Disrupt the Blood-Brain Barrier,” PLOS ONE 6 (11): e27877. doi: 10.1371, by Burgess, et al. reports using MRI guided focused ultrasound with microbubbles to temporarily open targeted regions of the BBB to allow entry of neural stem cells in animal models. Similarly, “Cellular Mechanisms Of The Blood-Brain Barrier Opening Induced By Ultrasound In Presence Of Microbubbles,” Ultrasound in Med. & Biol., Vol. 30, No. 7, pp. 979-989, 2004 by Sheikov, et al reports using ultrasound and microbubbles in animal models to open the BBB, but cautions tissue in the BBB can be damaged at 3W sonications. Likewise, “Noninvasive Localized Delivery Of Herceptin To The Mouse Brain By Mri-Guided Focused Ultrasound-Induced Blood-Brain Barrier Disruption,” PNAS, Vol. 103, No. 31, 11719-23 by Kinoshita, et al reports using ultrasound with microbubbles in animal models to disrupt BBB and deliver Herceptin (150 kDa) across the BBB, and notes the presence of microbubbles is required for consistent BBB opening. But it does not appear known to deliver microvesicles across the BBB or to use shockwaves to facilitate migration of microvesicles across the BBB.

Therapeutic use of shockwaves on the human body are known, but are taught to be dangerous and harmful to organs, particularly the human brain. See, e.g., U.S. Pat. No. 10,639,233 to Luc.

All publications identified herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Thus, there remains a need for systems and methods for facilitating or inducing delivery of stem cells or microvesicles to the brain in humans.

### SUMMARY OF THE INVENTION

The inventive subject matter provides apparatus, systems, and methods for delivering a structure to across the blood-brain barrier (BBB) of a (preferably human) patient. The structure is selected for therapeutic treatment of a patient condition, and is typically a microvesicle or a stem cell.

A method of delivering a microvesicle or stem cell to a patient's brain by facilitating crossing of the BBB is contemplated. A region of the patient's brain is targeted with a pressure device, typically via an MRI or CT scan navigation methodology. The pressure device is used to induce a pressure wave in the targeted region of the patient's brain and facilitate selective delivery of the structure to the region of the patient's brain. The structure is then administered to the patient's blood stream, spinal fluid space, or proximal to the patient's brain barrier. The structure, for example stem cell, then traverses the BBB at the targeted region.

The pressure device is used to generate a shockwave, a type of pressure wave. A shockwave differs from an acoustic or ultrasound wave in that a shockwave is a rapidly-rising (e.g., short rise period) and brief pressure wave administered in a repetitive fashion, in this case to the head of the patient. Indeed, the shockwave waveform is characterized by a rapid raise with an abrupt falloff of drop, and is taught in the art as dangerous when applied to the head or brain. Viewed from another perspective, while an acoustic or ultrasound wave is designed to be sinusoidal, a shockwave is not and is rather abrupt. As such, shockwaves are known to cause injury to the head and are medically prohibited for such therapeutic use. See, e.g., U.S. Pat. No. 10,639,233 to Lue, teaching dangers of applying shockwaves to various organs and the resulting harm or injury.

The stem cells or microvesicles can include, or can be made up of, a liposome, an exosome, whole or partial cells, or combinations thereof. The structures can also include, or can be made up of, nanoparticles, which in some, most, or all circumstances are completely synthetic (e.g., not naturally occurring, created by human intervention, etc.). Similarly, exosomes, stem cells, or other cells or cell products can be derived from a natural or expanded cell culture (e.g., extracted, concentrated, isolated, etc.), derived from full term placental tissue, derived from an umbilical tissue, or otherwise processed from natural cultures or biological sources. Nanoparticles or liposomes can be completely synthetic (e.g., requiring human intervention, combination, alteration, etc.), mostly synthetic, partially synthetic, or otherwise not entirely natural.

### BRIEF DESCRIPTION OF THE DRAWINGS

FIG. 1 depicts a flow chart of a method of the inventive subject matter.

### DETAILED DESCRIPTION

The inventive subject matter provides apparatus, systems, and methods for delivering a structure to across the blood-brain barrier (BBB) of a (preferably human) patient. The structure is selected for therapeutic treatment of a patient condition, and is typically a microvesicle or a stem cell.

A method of delivering a microvesicle or stem cell to a patient's brain by facilitating crossing of the BBB is contemplated. A region of the patient's brain is targeted with a pressure device, typically via an MRI or CT scan navigation methodology. The pressure device is used to induce a pressure wave in the targeted region of the patient's brain and facilitate selective delivery of the structure to the region of the patient's brain. The structure is then administered to the patient's blood stream, spinal fluid space, or proximal to the patient's brain barrier. The structure, for example stem cell, then traverses the BBB at the targeted region.

Targeting the region of the brain, or aiming the shockwave at the target, is typically done using coordinates form an imaging data set of the patient, whether taken for the purpose of performing the method on the patient or previously existing in the patient's medical record. An optical tracking device can be used to navigate coordinates plotted onto the subject's head, or frameless stereotactic methods can be used.

The pressure device is used to facilitate delivery of the structure, for example stem cell, across the BBB in a manner that is non-damaging and non-harmful to the BBB or brain tissue. For example, shockwaves applied to a targeted region of the brain can surprisingly be used to induce transcytosis delivery of microvesicles or stem cells across the BBB at the targeted region. However, the key consideration of the inventive subject matter is that shockwaves are applied to the BBB and brain in a manner unharmful to the targeted area, and migration of microvesicles or stem cells across the BBB are likewise induced or facilitated by shockwaves in an unharmful manner.

For example, the inventive subject matter is not designed to use on any microbubbles or other mechanisms that may damage or harm the BBB, for example tearing open the tight intercellular junctions that comprise the BBB, or causing permanent or long term (e.g., greater than 24 hours, 5 days, or 14 days) negative effects to the BBB or neighboring brain tissue. Viewed from another perspective, the inventive subject matter is designed to not use or rely on methods of disruption or damage of the BBB (e.g., use of microbubbles), and does not otherwise rely on disruption of the BBB to induce or facilitate microvesicles, stem cells, or other structures to cross the BBB.

Generally a plurality of microvesicles, stem cells, or other structures are administered to the patient, whether of the same type (e.g., all exosome, all liposome, all hybrid exosome/liposome, all nanoparticle, all stem cells, etc.), of the same type but different versions of the type (e.g., exosomes A and B, liposomes A and B, stem cells A and B, etc.), or of combinations of types or versions of types (e.g, stem cells with microvesicles, stem cells with exosomes, stem cells with liposomes, stem cells with nanoparticles, etc.). Surprisingly applying shockwaves to a targeted region of the patient's brain enhances the actual traversal of microvesicles, stem cells, and other related structures across or through the BBB at the targeted region, in some cases more than 5%, 100%, 1000% or greater compared to administration of microvesicles, stem cells, or other related structures without shockwave treatment, whether preceding, during, or after administration of the structures.

Microvesicles, stem cells, or other structures can be administered to the patient at the same time, in an overlapping manner, or in a step-wise manner. Likewise, structures can be administered to the patient before, during, or shortly after (e.g., within 10, 30, 60, or 90 seconds, within 1, 2, 3, 5, 10, or 15 minutes) the region of the patient's brain is targeted with the pressure device, the application of the pressure device to the patient to induce a pressure wave, or conditioning of the BBB by pressure wave to enable the structure to traverse the BBB.

Stem cells can include embryonic stem cells, induced pluripotent stem cells, tissue stem cells, hematopoietic stem cells, mesenchymal stem cells, neural stem cells, or epithelial stem cells. The stem cells can be natural, naturally derived, engineered, doped, or otherwise altered.

The targeted region of the patient's brain is typically associated with a condition (e.g., health, mental, physical, etc.) of the patient that would benefit from or require alteration, optimization, remediation, therapy, or improvement. For example, patient conditions can be associated with a disease, dementia, a learning disorder, an anxiety disorder, a motor disorder, a consciousness disorder, a movement disorder, an attention disorder, a stroke, a vascular disease, Alzheimer's disease or other progressive potentially degenerative conditions, Parkinson's disease, multiple sclerosis, cancer, schizophrenia, depression, anxiety disorder, developmental disorder, substance abuse, and traumatic brain injury. Likewise, the region of the patient's brain for targeting by shockwaves or microvesicles includes the frontal lobe, parietal lobe, occipital lobe, temporal lobe, hippocampus, hypothalamus, brain stem, cerebellum amygdala, corticospinal tract, thalamus, substantia nigra, basal ganglia, a tumor, a lesion, necrotic tissue, Heschl's gyrus, Brodmann area 25, spinal cord, a point of injury, or any other region of a patient's brain associated with a condition of the patient or altering the condition of the patient.

Pressure devices generating the shockwaves are typically electrohydraulic, electromagnetic, and piezoelectric, or combinations thereof. In some embodiments, more than one pressure device, or more than one type of pressure device, can be used, whether concurrently or sequentially. Shockwaves are typically induced for at least 1 minute, at least 10 minutes, or up to two hours, and can be of different types (e.g., different peaks and drops, duration, etc.). Further, the period of induced or facilitated migration of microvesicles across the BBB (e.g., via transcytosis) is temporary and can last 1, 2, 5, 10 minutes up to 1, 2, 3, or many hours after applying shockwaves.

While it is contemplated the inventive subject matter is applicable to any condition (e.g., disease, disorder, characteristic, etc) and region of the brain, preferred conditions for treatment, and regions of the brain for targeting, include those listed in Table 1.

TABLE 1

| Condition | Region of the Brain |
|---|---|
| Alzheimer's disease: | Hippocampus and surrounding cortex |
| Parkinson's disease: | Substantia nigra and basal ganglia |
| Vascular dementia: | Diffusely throughout the brain |
| MS: | Proximal to MS lesions |
| Cancer: | Proximal to tumor and necrotic tissue |
| Schizophrenia: | Frontal lobe and Heschl's gyrus |
| Depression: | Frontal lobe and Brodmann area 25 |
| Substance abuse: | Diffusely throughout the cortex but likely not |
| | in subcortical structures |
| Traumatic Brain | Proximal to area of injury |
| Injury: | |

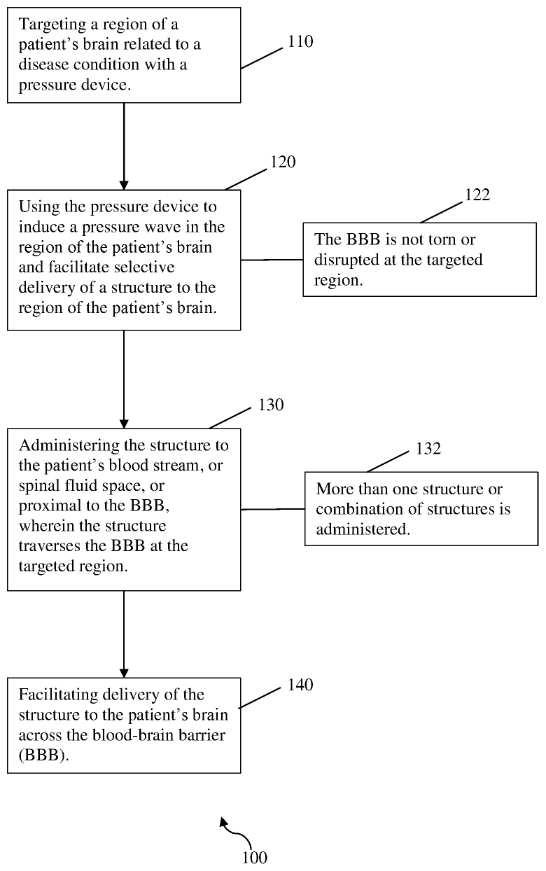

FIG. 1 depicts flow chart 100 of a method of the inventive subject matter, including steps 110, 120, 130, and 140, and optional steps 122 and 132. It is contemplated that one or more of steps 110, 120, 130, or 140 can be repeated, performed in sequential order, or performed in non-sequential order.

Various objects, features, aspects, and advantages of the inventive subject matter will become more apparent from the following detailed description of preferred embodiments, along with the accompanying drawing figures in which like numerals represent like components.

The inventive subject matter provides apparatus, systems, and methods for comparative analysis of tissue and organ scans between patients or groups of patients without sensitivity to patient-specific or scanner specific characteristics, including prediction, diagnosis, prognosis, tracking, and treatment guidance.

The following description includes information that may be useful in understanding the present invention. It is not an admission that any of the information provided herein is prior art, necessary, or relevant to the presently claimed invention, or that any publication specifically or implicitly referenced is prior art.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

Unless the context dictates the contrary, all ranges set forth herein should be interpreted as being inclusive of their endpoints, and open-ended ranges should be interpreted to include commercially practical values. Similarly, all lists of values should be considered as inclusive of intermediate values unless the context indicates the contrary.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A method of facilitating delivery of a structure to a patient's brain across the blood-brain barrier (BBB), comprising:
targeting a region of the patient's brain with a pressure device;
using the pressure device to induce a pressure wave in the region of the patient's brain and facilitate selective delivery of the structure to the region of the patient's brain; and
administering the structure to the patient's blood stream, or spinal fluid space, or proximal to the BBB, wherein the structure traverses the BBB at the targeted region;
wherein the structure is one of a microvesicle or a stem cell.

2. The method of claim 1, wherein the pressure wave is a shockwave administered in a repetitive fashion.

3. The method of claim 1, wherein the microvesicle is one of a liposome, an exosome, or a nanoparticle.

4. The method of claim 1, wherein the region of the patient's brain is targeted via imaging data based on an MRI or a CT scan navigation.

5. The method of claim 1, wherein delivery of the structure is selectively facilitated by transcytosis.

6. The method of claim 1, wherein no microbubble is administered to the patient, the BBB at the targeted region is not torn during performance of the method, or the BBB is not disrupted at the targeted region.

7. The method of claim 1, further comprising administering a plurality of microvesicles or stem cells, and wherein at least 50% of the plurality of microvesicles or stem cells traverse the BBB at the targeted region.

8. The method of claim 1, wherein the region of the patient's brain is associated with a health, physical, or mental condition sought to be altered.

9. The method of claim 8, wherein the condition is associated with one of the following: a disease, dementia, a learning disorder, an anxiety disorder, a motor disorder, a consciousness disorder, a movement disorder, an attention disorder, a stroke, a vascular disease, Alzheimer's disease or other progressive potentially degenerative conditions, Parkinson's disease, multiple sclerosis, cancer, schizophrenia, depression, anxiety disorder, developmental disorder, substance abuse, and traumatic brain injury.

10. The method of claim 1, wherein the region of the patient's brain is selected from the group consisting of frontal lobe, parietal lobe, occipital lobe, temporal lobe, hippocampus, hypothalamus, brain stem, cerebellum amygdala, corticospinal tract, thalamus, substantia nigra, basal ganglia, a tumor, a lesion, necrotic tissue, Heschl's gyrus, Brodmann area 25, spinal cord, and a point of injury.

11. The method of claim 3, wherein the structure is derived from a natural cell culture, an expanded cell culture, a full term placental tissue, or an umbilical tissue.

12. The method of claim 1, wherein the pressure device is one of electrohydraulic, electromagnetic, or piezoelectric.

13. The method of claim 1, wherein the pressure wave is induced for at least 10 minutes.

14. The method of claim 13, wherein the step of administering the structure occurs after inducing the pressure wave.

15. The method of claim 1, wherein selective delivery of the structure to the region of the patient's brain is facilitated for between 10 minutes and 1 hour.

16. The method of claim 1, selective delivery of the structure to the region of the patient's brain does not harm or degrade the BBB.

17. The method of claim 3, wherein one of the nanoparticle or liposome is synthetic.

18. The method of claim 9, wherein the structure is selected to alter the condition.

19. The method of claim 2, wherein the shockwave does not tear or disrupt the BBB at the targeted region.

20. The method of claim 1, wherein the pressure device is a first pressure device and a pressure wave is a first pressure wave, further comprising using a second pressure device to induce a second pressure wave in a region of the patient's brain.
